(19) United States
(12) Patent Application Publication
(10) Pub. No.: US 2008/0188664 A1
(43) Pub. Date: Aug. 7, 2008

(54) PROCESS FOR PREPARING MONTELUKAST SODIUM CONTAINING CONTROLLED LEVELS OF IMPURITIES

(75) Inventors: Michael Brand, Ra'anana (IL); Alex Weisman, Kiriyat Ekron (IL); Yael Gafni, Rosh Ha'ayin (IL); Lior Zelikovitch, Mazkeret Batya (IL); Guy Davidi, Even Yehuda (IL); Efrat Manoff, Lehavim (IL)

Correspondence Address: MARSHALL, GERSTEIN & BORUN LLP, 233 S. WACKER DRIVE, SUITE 6300, SEARS TOWER, CHICAGO, IL, 60606, US

(73) Assignee: CHEMAGIS LTD., Bnei Brak (IL)

(21) Appl. No.: 11/972,113

(22) Filed: Jan. 10, 2008

Related U.S. Application Data
(60) Provisional application No. 60/884,927, filed on Jan. 15, 2007.

(51) Int. Cl. C07D 215/00

(52) U.S. Cl. 546/180

(57) ABSTRACT

The present invention is directed to a process of preparing montelukast or a salt thereof with minimal amounts of impurities, such as a dehydration impurity (compound (VI)) or a cyclic ether impurity (compound (VIII)).

### FIELD OF THE INVENTION

The invention relates to the chemistry of the drug montelukast sodium, and more particularly to an improved process for preparing montelukast sodium containing a low level of a dehydration impurity, which results from elimination of the tertiary alcohol group of montelukast to form a styrenyl moiety.

### BACKGROUND OF THE INVENTION

(R-(E))-1-(((1-(3-(2-(7-chloro-2-quinolinyl)ethenyl)phenyl)-3-(2-(1-hydroxy-1-methylethyl)phenyl)propyl)thio)methyl)cyclopropaneacetic acid sodium salt, also known by the name montelukast sodium, is represented by the structural formula (I) below:

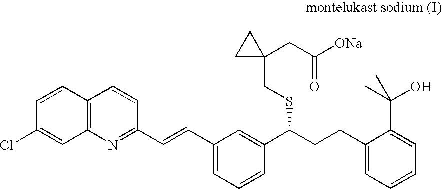

Montelukast sodium is a leukotriene antagonist, and is useful as an anti-asthmatic, anti-allergic, anti-inflammatory and cytoprotective agent. Montelukast sodium is currently indicated for the treatment of allergic rhinitis and asthma.

Montelukast sodium, formulated as tablets (containing 10.4 mg montelukast sodium), chewable tablets (containing 4.2 or 5.2 mg montelukast sodium) or oral granules (in a packet containing 4.2 mg montelukast sodium), typically is given once daily to a patient for the treatment of asthma and seasonal allergic rhinitis. Montelukast sodium is marketed in the United States and other countries by Merck & Co., Inc. under the trade name SINGULAIR®.

Montelukast sodium and related compounds were first disclosed in European Patent No. 480 717. The synthesis of montelukast sodium, as taught in EP 480 717, is depicted in Scheme 1 and involves reacting compound (II) with methanesulfonyl chloride (MsCl) to obtain compound (III). Compound (III) then is reacted with compound (IV) to obtain compound (V). Compound (V) is hydrolyzed to form montelukast acid, which then can be converted into the corresponding sodium salt, i.e., compound (I).

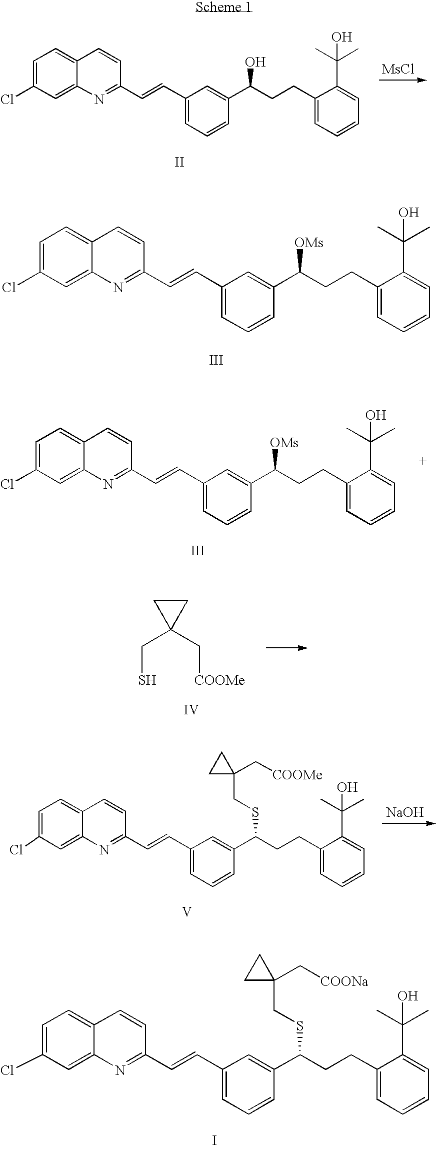

U.S. Pat. No. 5,523,477 describes the formation of montelukast and its subsequent conversion into the dicyclohexyl ammonium salt, which is converted to montelukast sodium.

U.S. Pat. No. 5,614,632 describes a method of preparing crystalline montelukast sodium, which involves the preparation of the dilithium dianion of 1-(mercaptomethyl)cyclopropaneacetic acid (compound (VII)), using butyl lithium, followed by condensation thereof with compound (III) to yield montelukast acid as a viscous oil, after workup. The resulting montelukast acid is converted, via the corresponding dicyclohexyl ammonium salt, into crystalline montelukast sodium.

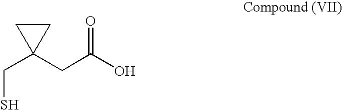

An extra purification step via the dicyclohexyl ammonium salt, which is disclosed in U.S. Pat. Nos. 5,523,477 and 5,614,632, is required due to the difficulties encountered in obtaining crystalline montelukast sodium. Thus, the crude montelukast acid is purified via the dicyclohexylamine salt by reacting the acid with dicyclohexylamine in ethyl acetate, followed by addition of hexanes to effect crystallization of the dicyclohexylamine salt, or by the crystallization from toluene/heptane. It is mentioned in U.S. Pat. No. 5,614,632 that crystalline montelukast dicyclohexylamine salt offers an efficient method for the purification of montelukast, which obviates the need to use chromatographic purification.

According to the regulatory authorities, such as the FDA, EU authorities, and the ICH (The International Conference on Harmonisation of Technical Requirements for Registration of Pharmaceuticals for Human Use), a drug manufacturer must submit data demonstrating that the product intended for marketing complies with the regulations with regard to the content of impurities. The content of an unidentified impurity cannot exceed 0.1% by weight, while the amount of a known impurity cannot exceed 0.15%. The drug manufacturer usually submits analytical data to the regulatory authority demonstrating that the content of each impurity is in accordance with the regulations. The regulatory authority checks the submitted data in order to ensure that the drug is free of impurities and is suitable for marketing.

U.S. Patent Application Publication No. 2006/0194839 discloses a process for converting montelukast dicyclohexylamine salt into the acid form by treatment with dilute solution of a weak acid, indicating it is a “somewhat delicate” step because impurities, such as (R-(E))-1-(((1-(3-(2-(7-chloro-2-quinolinyl)ethenyl)-phenyl)-3-(2-(1-methylethenyl)phenyl)propyl)thio)methyl)-cyclopropaneacetic acid (compound (VI)), may be formed during the treatment with acid. This impurity is derived from dehydration of the tertiary alcohol group and “is difficult to remove.”

Dufresne et al., J. Org. Chem. 61:8518-8525 (1996) discloses that dihydroxylation of styryl analogs of montelukast, such as compound (VI), with osmium tetroxide/4-methylmorpholine N-oxide or with asymmetric dihydroxylation catalysts, such as the AD-mix catalysts, failed to give any desired material, i.e., montelukast acid. Consequently, compound (VI) is a relatively stable compound and does not react easily.

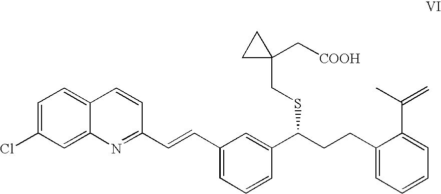

Because impurities, such as compound (VI), must be limited for a viable chemical synthesis of montelukast as an active pharmaceutical ingredient, and the difficulty encountered in removing such impurities, there exists a need for an improved process for preparing montelukast and salts thereof containing low levels of these impurities, i.e., less than 0.15%, as required by the regulatory authorities.

### SUMMARY OF THE INVENTION

Disclosed herein are processes for synthesizing montelukast or salts thereof having controlled amounts of impurities, such as the dehydration impurity, compound (VI), and compound (VIII), a cyclic ether.

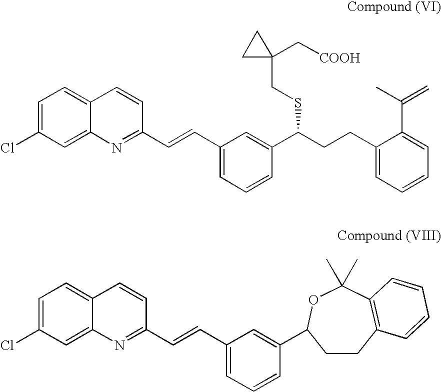

More particularly, disclosed herein is an improved process for the synthesis of montelukast or salts thereof comprising reacting compound (II) with methanesulfonyl chloride, wherein the molar ratio of methanesulfonyl chloride to compound (II) is less than 2:1, but greater than 1:1.

Thus, in one embodiment of the present invention, there is provided an improved process for preparing montelukast acid or salts thereof, having low levels of compound (VI), comprising the steps of: (a) admixing compound (II) and methanesulfonyl chloride to obtain compound (III); (b) admixing compound (III) and compound (VII) to obtain montelukast or a salt thereof; and (c) admixing compound (III) and compound (VII), optionally in the presence of a base, to obtain montelukast or a salt thereof, and wherein the amount of compound (VI) produced is less than 0.15%, less than 0.12%, or less than 0.1% by weight.

The molar ratio of methanesulfonyl chloride to compound (II) is about 1.1:1 to about 1.5:1. In some cases, the molar ratio of methanesulfonyl chloride to compound (II) is about 1.15:1 to about 1.45:1, about 1.2:1 to about 1.45:1, or about 1.35:1 to about 1.45:1. Molar ratios greater than about 1.5:1 typically produce greater amounts of impurities, such as compound (VI), while molar ratios less than about 1.1:1 do not produce a high enough yield of compound (III).

The process also comprises adding a base to compound (II) and cooling, prior to addition of the methanesulfonyl chloride. The mixture of the base and compound (II) is typically cooled to below about 20° C., but can be cooled to below about 10° C., below about 0° C., or at or below about −10° C. The base is typically an organic base, and more specifically, an amine. Amines contemplated for use in the process include, but are not limited to, triethylamine, tripropylamine, triisopropylamine, tributylamine, triisobutylamine, N,N-diisopropylethylamine, N,N-dimethylaniline, and mixtures. In a specific embodiment, the base comprises N,N-diisopropylethylamine (DIEA).

The molar ratio of base to compound (II) is typically less than or equal to 2:1. In various embodiments, the molar ratio of base to compound (II) is about 1.2:1 to about 2:1, about 1.25:1 to about 1.8:1, about 1.3:1 to about 1.5:1, or about 1.35:1 to about 1.45:1. Molar ratios of base to compound (II) above about 2:1 can lead to greater production of undesired impurities, such as compound (VI), while molar ratios of base to compound (II) below about 1.2:1 are less efficient for maximizing product yield.

The methansulfonyl chloride can be added to the cooled mixture of base and compound (II). The addition can be dropwise or at least two portions. The methanesulfonyl chloride can be added in three portions, four portions, five portions, or six portions.

In another embodiment, the admixture of compound (III) and compound (VII) further comprises mixing compound (VII) and an organic solvent; adding a base to the mixture; and then adding compound (III), where compound (III) can be added dropwise or in at least two portions. The organic solvent can be N,N-dimethylformamide, N,N-dimethylacetamide, dimethyl sulfoxide, N-methyl-2-pyrrolidone, tetrahydrofuran, 2-methyltetrahydrofuran, acetonitrile, acetone, or mixtures thereof. In some cases, the organic solvent comprises N-methyl-2-pyrrolidone.

The base is typically an inorganic base. Specific bases contemplated for use include, but are not limited to, lithium hydroxide, potassium hydroxide, sodium hydroxide, sodium carbonate, potassium carbonate, sodium bicarbonate, potassium bicarbonate, and mixtures thereof. In one embodiment, the base comprises sodium hydroxide. The molar ration of base to compound (III) is about 1.5:1 to about 2.5:1.

A cosolvent, such as water, can be added to the mixture of compound (VII) and the organic solvent to form a suspension, prior to the addition of compound (III). The amount of cosolvent is typically at least 1% by volume of the organic solvent, and can be about 3%, about 5%, or about 10%. In some embodiments, the cosolvent is about 5% by volume of the organic solvent.

In another embodiment, the process further comprises admixing an organic acid and the reaction product of compound (III) and (VII) (i.e., step (c)) to obtain montelukast acid. The organic acid of this step can be any acid compatible with the reaction conditions, but typically is selected from acetic acid, propionic acid, oxalic acid, benzoic acid, maleic acid, malonic acid, fumaric acid, tartaric acid, malic acid, citric acid, and mixtures thereof. In a specific embodiment, the organic acid comprises tartaric acid.

In still another embodiment, the process further comprises admixing an amine and the reaction product of compound (III) and (VII) (i.e., step (c)) to obtain an ammonium salt of montelukast. Non-limiting examples of amines that can be used include cyclohexylamine, cyclopentylamine, cycloheptylamine, cyclooctylamine, cyclododecylamine, phenethylamine, and mixtures thereof. In one case, the amine is cyclooctylamine. In some cases, this process can also include seeding the admixture with a crystal of the ammonium salt of montelukast, and/or purifying the ammonium salt of montelukast. Purifying can be any means to remove impurities from the ammonium salt of montelukast, including, but not limited to, crystallizing the ammonium salt of montelukast, using chromatography or other separation techniques, extracting the ammonium salt of montelukast from impurities, filtering the ammonium salt of montelukast, or mixtures of any two or more of these techniques.

Crystallizing the ammonium salt of montelukast typically comprises adding an organic solvent to the ammonium salt of montelukast in order to promote crystallization. Typical organic solvents used include, but are not limited to, methanol, methyl acetate, ethyl acetate, n-propyl acetate, isopropyl acetate, n-butyl acetate, isobutyl acetate, toluene, and mixtures thereof. In some cases, the organic solvent comprises toluene or toluene having up to 5% methanol.

In some embodiments, the ammonium salt of montelukast is converted to the sodium salt of montelukast (i.e., montelukast sodium). Therefore, the process further comprises (1) admixing the ammonium salt, an acid, an organic solvent, and water; (2) separating the water; (3) optionally evaporating at least a portion of the organic solvent; (4) adding a base and water; (5) separating the organic solvent; and (6) drying the resulting aqueous mixture to obtain montelukast sodium. The drying can be via spray-drying. The organic acid used can be any organic acid compatible with the process, but is typically acetic acid, propionic acid, oxalic acid, benzoic acid, maleic acid, malonic acid, fumaric acid, tartaric acid, malic acid, citric acid, or mixtures thereof. In a specific embodiment, the organic acid is tartaric acid.

### DETAILED DESCRIPTION

It has been found that substantial quantities of impurities, e.g., compounds (VI) and (VIII), can be formed during the synthesis of montelukast and salts thereof.

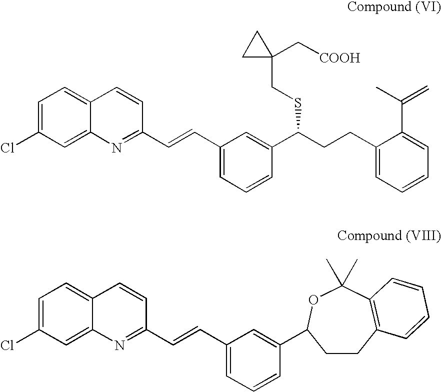

Two steps in the process that are particularly susceptible to forming impurities are the reaction of compound (II) and methanesulfonyl chloride to form compound (III) and the reaction of compound (III) with compound (IV) or compound (VII). In order to satisfy the requirements of various regulatory bodies for minimal impurities in an active pharmaceutical ingredient (API), it is important to synthesize APIs using a process that minimizes the amount of impurities produced during the various synthetic steps. One of the main impurities of montelukast or its salts is compound (VI), a dehydration impurity which is difficult to remove once it has been formed. Therefore, the disclosed processes are directed to minimizing the amount of impurities formed.

Scheme 2, below, outlines the general processes disclosed herein, which produces minimal levels of impurities such as the dehydration impurity, compound (VI), and/or the cyclic ether impurity, compound (VIII).

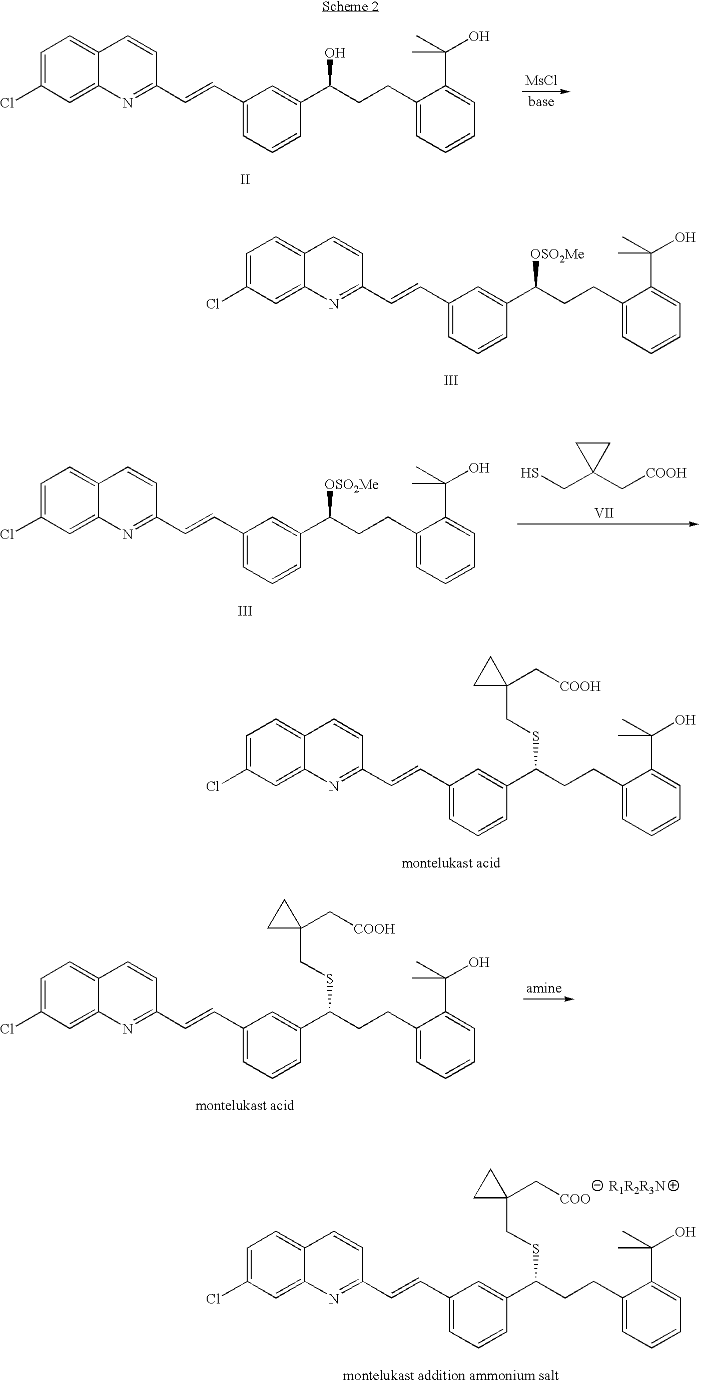

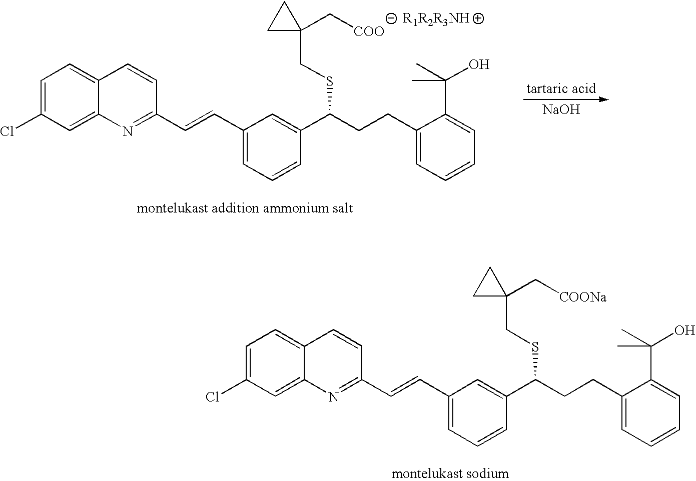

By controlling the molar ratios of base and methanesulfonyl chloride to compound (II), the formation of the dehydration impurity compound (VI) can be minimized, in comparison to previously disclosed processes of preparing montelukast or salts thereof. Table 1 shows the comparison of a process using a large excess of methanesulfonyl chloride and base with respect to compound (II) (Example 1) to the process using smaller molar excesses of methanesulfonyl chloride and base with respect to compound (II) (Example 2). The amount of compound (VI) produced when using a larger excess of methanesulfonyl chloride and base (e.g., Example 1) is much higher than that produced when using a smaller molar excess (e.g., Examples 2 and 3).

TABLE 1

| | Example 1 | | Example 2 | | Example 3 | |
|---|---|---|---|---|---|---|
| Reaction Conditions | | | | | | |
| Compound (II) | 1 | equiv. | 1 | equiv. | 1 | equiv. |
| Base (diisopropyl- | 2.27 | equiv. | 1.4 | equiv. | 1.42 | equiv. |
| ethylamine) | | | | | | |
| Methanesulfonyl | 1.97 | equiv. | 1.2 | equiv. | 1.22 | equiv. |
| Chloride | | | | | | |
| Temperature | −20° | C. | −20° | C. | 0° | C. |
| Reaction time | 2 | hours | 2 | hours | 3 | hours |
| Results | | | | | | |

| | | | |
|---|---|---|---|
| % of compound (II) | 1% | 5% | 0.74% |
| at the end of the | | | |
| reaction | | | |
| % of compound (VI) | 1.5% | 0.5% | 0.3% |
| at the end of the | | | |
| reaction | | | |
| Reaction yield | 70% | 68% | 70% |
| Purity of crude | 90.4% | 98% | 97.7% |
| cyclooctyl ammonium | | | |
| salt | | | |
| % of compound (VI) | 1.7% | 0.23% | 0.25% |
| in the crude | | | |
| cyclooctyl ammonium | | | |
| salt | | | |
| Purity of the | 97% | 99% | 99.5% |
| crystallized | | | |
| cyclooctyl ammonium | | | |
| salt | | | |
| % of compound (VI) | 1.4% | 0.1% | 0.17% |
| in the crystallized | | | |
| cyclooctyl ammonium | | | |
| salt | | | |

Thus, one embodiment provides an improved process for preparing montelukast acid or salt thereof, having low levels of compound (VI), comprising the steps of: (a) admixing compound (II) and methanesulfonyl chloride to obtain compound (III); and (b) admixing compound (III) and compound (VII), optionally in the presence of a base, to obtain montelukast or a salt thereof, wherein the amount of compound (VI) produced is less than 0.15%, less than 0.12%, or less than 0.1% by weight.

The molar ratio of methanesulfonyl chloride to compound (II) is about 1.1:1 to about 1.5:1. In some cases, the molar ratio of methanesulfonyl chloride to compound (II) is about 1.15:1 to about 1.45:1, about 1.2:1 to about 1.45:1, or about 1.35:1 to about 1.45:1. Molar ratios greater than about 1.5:1 typically produce greater amounts of impurities, such as compound (VI), while molar ratios less than about 1.1:1 do not produce a high enough yield of compound (III).

The process also comprises adding a base to compound (II) and cooling, prior to addition of the methanesulfonyl chloride. The mixture of the base and compound (II) is typically cooled to below about 20° C., but can be cooled to below about 10° C., below about 0° C., or at or below about −10° C. The base is typically an organic base, and in specific embodiments, is an amine. Amines contemplated for use in the process include, but are not limited to, triethylamine, tripropylamine, triisopropylamine, tributylamine, triisobutylamine, N,N-diisopropylethylamine, N,N-dimethylaniline, and mixtures thereof. In a specific embodiment, the base comprises N,N-diisopropylethylamine (DIEA).

The molar ratio of base to compound (II) is typically less than or equal to 2:1. In various embodiments, the molar ratio of base to compound (II) is about 1.2:1 to about 2:1, about 1.25:1 to about 1.8:1, about 1.3:1 to about 1.5:1, or about 1.35:1 to about 1.45:1. Molar ratios of base to compound (II) above about 2:1 can lead to greater production of undesired impurities, such as compound (VI), while molar ratios of base to compound (II) below about 1.2:1 are less efficient for maximizing product yield.

The methansulfonyl chloride can be added to the cooled mixture of base and compound (II). The addition can be dropwise or at least two portions. The methanesulfonyl chloride can be added in three portions, four portions, five portions, or six portions.

In another embodiment, the admixture of compound (III) and compound (VII) further comprises mixing compound (VII) and an organic solvent; adding a base to the mixture; and then adding compound (III), where compound (III) can be added dropwise or in at least two portions. The organic solvent can be N,N-dimethylformamide, N,N-dimethylacetamide, dimethyl sulfoxide, N-methyl-2-pyrrolidone, tetrahydrofuran, 2-methyltetrahydrofuran, acetonitrile, acetone, or mixtures thereof. In some cases, the organic solvent comprises N-methyl-2-pyrrolidone.

Compound (VII) can be mixed with a base prior to reaction with compound (III). Typically, the base is an inorganic base. Non-limiting examples of bases include lithium hydroxide, potassium hydroxide, sodium hydroxide, sodium carbonate, potassium carbonate, sodium bicarbonate, potassium bicarbonate, and mixtures thereof. The ratio of base to compound (VII) is typically at least about 1.1:1, but can be about 1.5:1 to about 2.5:1, or about 1.8:1 to about 2.1:1. Compound (VII) and the base typically are mixed in a polar organic solvent. Non-limiting examples of organic solvents suitable for this step include N,N-dimethylformamide (DMF), N,N-dimethylacetamide (DMA), dimethyl sulfoxide (DMSO), N-methyl-2-pyrrolidone (NMP), tetrahydrofuran (THF), 2-methyltetrahydrofuran, acetonitrile, acetone, and mixtures thereof. Typically, the polar organic solvent is NMP. The organic solvent can be mixed with a cosolvent, such as water. The amount of cosolvent is at least 1% by volume, and can be about 3% to about 10%, or about 4% to about 6% by volume.

Not bound by theory, it is postulated that a polar solvent, e.g., NMP with optional addition of about 5% water, efficiently dissolves the dianion of compound (VII) (e.g., a disodium dianion) and allows for better reaction between compound (VII) and compound (III).

Montelukast acid can be isolated from the reaction of compound (VII) and compound (III) by adding an acid to the mixture. The acid can be either an inorganic acid or an organic acid. Preferably, the acid is an organic acid selected from the group consisting of acetic acid, propionic acid, oxalic acid, benzoic acid, maleic acid, malonic acid, fumaric acid, tartaric acid, malic acid, citric acid, and combinations thereof, and preferably comprises tartaric acid.

A process for producing an ammonium salt of montelukast is disclosed herein. The process comprising admixing an amine and the reaction product of compound (VII) and compound (III) to obtain an ammonium salt of montelukast, and optionally purifying the ammonium salt of montelukast. Nonlimiting examples of an amine include cyclohexylamine, cyclopentylamine, cycloheptylamine, cyclooctylamine, cyclododecylamine, and phenethylamine.

An organic solvent can be added to the reaction mixture prior to purifying the ammonium salt of montelukast. The organic solvent can be methyl acetate, ethyl acetate, n-propyl acetate, isopropyl acetate, n-butyl acetate, isobutyl acetate, chloroform, dichloromethane, toluene or mixtures thereof. Preferably, the organic solvent comprises toluene.

Purifying can be any means to remove impurities from the ammonium salt of montelukast, including, but not limited to, crystallizing the ammonium salt of montelukast, using chromatography or other separation techniques, extracting the ammonium salt of montelukast from impurities, filtering the ammonium salt of montelukast, or mixtures of any two or more of these techniques. When crystallizing is used, the mixture can optionally be seeded with a crystal of the ammonium salt of montelukast.

Crystallizing the ammonium salt of montelukast typically comprises adding an organic solvent to the ammonium salt of montelukast in order to promote crystallization. Typical organic solvents used include, but are not limited to, methanol, methyl acetate, ethyl acetate, n-propyl acetate, isopropyl acetate, n-butyl acetate, isobutyl acetate, toluene, and mixtures thereof. In some cases, the organic solvent comprises toluene or toluene having up to 5% methanol.

Further disclosed herein is a process for preparing the sodium salt of montelukast from an ammonium salt. The process further comprises (1) admixing the ammonium salt, an acid, an organic solvent, and water; (2) separating the water; (3) optionally evaporating at least a portion of the organic solvent; (4) adding a base and water; (5) separating the organic solvent; and (6) drying the resulting aqueous mixture to obtain montelukast sodium. The drying can be via spray-drying. The organic acid used can be any organic acid compatible with the process, but is typically acetic acid, propionic acid, oxalic acid, benzoic acid, maleic acid, malonic acid, fumaric acid, tartaric acid, malic acid, citric acid, or mixtures thereof. In a specific embodiment, the organic acid is tartaric acid. Non-limiting examples of the organic solvent include methyl acetate, ethyl acetate, n-propyl acetate, isopropyl acetate, n-butyl acetate, isobutyl acetate, chloroform, dichloromethane, toluene and mixtures thereof. Preferably, the organic solvent comprises ethyl acetate.

Also disclosed herein is a process for preparing compound (VI). The process comprises the steps of
(a) admixing montelukast sodium and an organic solvent;
(b) heating the admixture of step (a) to afford a solution;
(c) adding an acid to the solution, wherein the molar ratio of acid to montelukast sodium is at least about 1.1:1;
(d) adding water to the product of step (c);
(e) optionally seeding the mixture of step (d) with a crystal of compound (VI);
(f) crystallizing compound (VI) from the mixture of step (d) or step (c);
(g) optionally isolating the compound (VI) crystals; and
(h) optionally re-crystallizing the compound (VI) crystals from an organic solvent. Non-limiting examples of the organic solvent include toluene, xylenes, ethyl acetate, methyl ethyl ketone, methyl isobutyl ketone, isopropyl alcohol, and mixtures thereof. Toluene is a preferred organic solvent. The acid used in the process for preparing compound (VI) can be either an inorganic or organic acid. Preferably, the acid is an organic acid. Non-limiting examples of organic acids include acetic acid, propionic acid, oxalic acid, benzoic acid, maleic acid, malonic acid, fumaric acid, tartaric acid, malic acid, citric acid, and combinations thereof. Preferably, the organic acid comprises maleic acid.

Compound VI prepared using the disclosed processes is produced in at least about 70% yield. The purity is at least 98.5%, and can be more than 99%, or greater than 99.5% (as determined by HPLC).

Reference is now made to the following examples, which together with the above descriptions, illustrate the invention in a non limiting fashion. Additional objects, advantages, and novel features of the present invention will become apparent to one ordinarily skilled in the art upon examination of the following examples, which are not intended to be limiting.

### EXAMPLES

### Example 1

### Preparation of Montelukast Sodium

A three-necked flask equipped with a thermometer, a nitrogen inlet and a magnetic stirrer was charged at room temperature with 9 g (0.0198 moles) of 2-(2-(3(S)-(3-(7-chloro-2-quinolinyl)ethenyl)phenyl)-3-(hydroxylpropyl)phenyl)-2-propanol (compound (II)) in 48 mL of anhydrous THF under stirring and cooled to about −20° C. N,N-diisopropylethylamine (DIPEA) (7.8 mL; 0.045 moles) was added in portions followed by addition of 3 mL (0.039 moles) of methanesulfonyl chloride in portions, and stirring was maintained at about −20° C. for about 2 hours. An aliquot was checked by HPLC, which contained less than 1% of the starting material, and about 1.5% of compound (VI) in the reaction mixture. The cold suspension containing the product 2-(2-(3S)-(3-(7-chloro-2-quinolinyl)ethenyl)phenyl)-3-(methanesulfonyloxypropyl)phenyl-2-propanol (compound (III)) was filtered at −20° C. and washed with cold anhydrous THF.

Another three-necked flask equipped with a thermometer, a nitrogen inlet and a magnetic stirrer was charged at room temperature with 7.3 g (0.04 moles) of 1-(mercaptomethyl)cyclopropaneacetic acid (compound (VII)) and 48 mL of N-methyl-2-pyrrolidone (NMP) under stirring and under nitrogen atmosphere to obtain a solution. Sodium hydroxide flakes (0.0112 moles; 4.5 g) was added in one portion at room temperature followed by addition of 2.4 mL of water, and stirring was maintained for 1 hour to afford a suspension. A solution of compound (III) in about 50 mL THF, at −20° C., was added in portions to the solution of compound (VII) at room temperature. After completing the addition, the mixture was stirred for 2 hours and reaction completion was checked by HPLC. Ethyl acetate (130 mL) and 5% sodium chloride solution (130 mL) were added to the reaction mixture, and the mixture was stirred for 20 minutes.

The layers were separated and the upper organic layer was washed with 130 mL of 5% sodium chloride solution, and the layers were separated. Tartaric acid (84 mL of 0.5 M solution) was added to the upper layer and the layers were separated. The upper layer was washed with 40 mL of water and again separated. The organic layer was distilled to dryness to afford an oily residue. Ethyl acetate (90 mL) was added to the residue and the mixture was distilled to dryness to afford 12.5 g of an oily residue. Ethyl acetate (90 mL) was added to the residue under stirring to obtain a solution. Cyclooctylamine (3.1 mL) was added and stirring was maintained for few minutes and the solution was seeded with crystalline montelukast acid cyclooctyl ammonium salt. Stirring was maintained at room temperature to afford a suspension, which was filtered off to obtain a cake. The cake was washed with ethyl acetate and dried at 40° C. in vacuum to afford 9.1 g of dry crude montelukast acid cyclooctyl ammonium salt in 70% yield. The HPLC purity was 90.4% (containing 1.7% compound (VII)). The crude montelukast acid cyclooctyl ammonium salt was crystallized from toluene to obtain a product having 97% purity and containing 1.4% of the methylene impurity. Ethyl acetate (25 mL) was added followed by addition of 17 mL of 0.5M tartaric acid solution. The mixture was stirred at room temperature for half an hour to afford a two phase system. The layers were separated and the upper ethyl acetate layer (containing the montelukast acid) was washed with 10 mL water. A solution of 0.15 g of solid sodium hydroxide in 25 mL of water was added in portions to the organic phase, and the layers were separated. The aqueous layer (containing the desired end product) was transferred into a three-necked flask equipped with nitrogen inlet, a stirrer, and a distillation head, and the solution was concentrated under vacuum at about 40° C. Acetonitrile was added and the mixture was allowed to cool to ambient temperature and seeded with crystalline montelukast sodium. The crystalline product was obtained by filtration and washed with acetonitrile.

### Example 2

### Preparation of Montelukast Sodium

A three-necked flask equipped with a thermometer, a nitrogen inlet and a magnetic stirrer was charged at room temperature with 9 g (0.0198 moles) of compound (II) in 48 mL of anhydrous THF under stirring and cooled to about −20° C. N,N-diisopropylethylamine (DIPEA; 4.8 mL; 0.028 moles) was added in portions followed by addition of 1.86 mL (0.024 moles) of methanesulfonyl chloride in portions, and stirring was maintained at about −20° C. for about 2 hours. A sample was withdrawn and checked by HPLC, which contained about 5% of the starting material, and about 0.5% of compound (VI) in the reaction mixture. The cold suspension containing the product compound (III), was filtered at −20° C. and the cake was washed with cold anhydrous THF.

Another three-necked flask equipped with a thermometer, a nitrogen inlet and a magnetic stirrer was charged at room temperature with 7.3 g (0.05 moles) of compound (VII) and 48 mL of NMP under stirring and under nitrogen atmosphere to obtain a solution. NaOH flakes (4.5 g; 0.0112 moles) was added in one portion at room temperature followed by addition of 2.4 mL of water, and stirring was maintained for 1 hour to afford a suspension. The solution of compound (III) in about 50 mL THF, which was kept at −20° C., was added in portions to the solution of compound (VII) at ambient temperature. After completing the addition, the mixture was stirred for half an hour and reaction completion was checked by HPLC. Toluene (130 mL) and 130 mL of a saturated sodium chloride solution (brine) were added to the reaction mixture and mixing was maintained for 20 minutes. The layers were separated and the organic layer was washed with 130 mL of brine, and the layers were separated. Tartaric acid (84 mL of 0.5 M solution) was added to the upper layer and the layers were separated. The upper layer was washed with 40 mL of water and again separated. The organic layer was distilled to dryness to afford an oily residue. Toluene (90 mL) was added to the residue and the mixture was distilled to dryness to afford 11 g of an oily residue. Toluene (90 mL) was added to the residue under stirring to obtain a solution. Cyclooctylamine (3.1 mL) was added and stirring was maintained for few minutes and the solution was seeded with crystalline montelukast acid cyclooctyl ammonium salt. Stirring was maintained at room temperature to afford a suspension, which was filtered off to obtain a cake. The cake was washed with toluene and dried at 40° C. in vacuum to afford 8.14 g of dry crude montelukast acid cyclooctyl ammonium salt in 68% yield. The HPLC purity was 98% (containing 0.23% compound (VI)). The crude montelukast cyclooctyl ammonium salt was crystallized from toluene containing about 2% of methanol to obtain a product having 99% purity and containing 0.1% compound (VI). Ethyl acetate (100 mL) was added followed by addition of 17 mL of 0.5M tartaric acid solution. The mixture was stirred at room temperature for half an hour to afford a two-phase system. The layers were separated and the ethyl acetate layer (containing the montelukast acid) was washed with 3×70 mL of water and concentrated by distillation to about half of its volume. A solution of 0.15 g of solid NaOH in 25 mL water was added in portions to the organic phase, and the layers were separated. The aqueous layer (containing the desired end product) was spray-dried to obtain the desired product having a purity of 99.8% and containing less than 0.1% of compound (VI) (according to HPLC).

### Example 3

### Preparation of Montelukast Sodium Salt

A dry reactor was charged at room temperature with 1.21 kg (2.66 mol) compound (II) and 6 L of anhydrous THF, cooled to 0° C. while stirring. DIEA (650 mL, 3.25 mol; 1.22 equiv.) was added in portions. A solution of methanesulfonyl chloride (250 mL, 3.25 mol, 1.22 equiv.) in 500 mL THF then was added to the stirred reaction mixture over a hour, while the temperature of the reaction mixture was maintained at or around 0° C. for about 3 hours. A sample of the reaction mixture was checked by HPLC, and indicated there was 0.74% of the starting material (compound (II)), and 0.3% of compound (VI). The cold reaction mixture then was filtered at 0° C. and the cake washed with 1 L anhydrous THF, then 2 L THF. The solution, containing the product (compound (III)) was then transferred to a clean and dry container.

Compound (VII) (480 g, 3.29 mol) was added to a clean and dry reactor at room temperature, followed by NMP (6.48 L) to form a solution. Next, NaOH (a 46% solution, 370 mL, 6.3 mol) was added in one portion at room temperature. Then, the solution of compound (III), prepared above, was added drop-wise at room temperature. Then, the reaction mixture was stirred at room temperature for 3 hours and monitored for completion via HPLC. Toluene (12.1 L) and 5% NaCl solution (17.4 L) were added to the reaction mixture and the resulting mixture was stirred for 20 minutes. The layers were separated and the organic layer was washed with water (5.7 L). Cyclooctylamine (420 mL) was added to the organic layer. Water was distilled out of the organic layer by azeotropic distillation over about 1.5 hours. The reactor was then cooled to about 40° C., and the solution seeded with crystalline montelukast cycloocyl ammonium salt. The solution was then cooled to room temperature, whereupon a suspension was formed. The suspension was filtered to obtain a cake. The cake was washed with toluene (550 mL) and dried to afford montelukast cycloocyl ammonium salt (1.13 g, 70% yield). The HPLC purity of the montelukast cycloocyl ammonium salt was 97.7%, and contained 0.25% compound (VI) and 0.46% compound (VIII). The montelukast cycloocyl ammonium salt then was crystallized from toluene having about 2% methanol to obtain montelukast cycloocyl ammonium salt in 85% yield having 99.5% purity and having 0.17% compound (VI) and 0.03% compound (VIII). The product was re-crystallized to obtain 99.7% purity and 0.17% compound (VI) and no detectable compound (VIII).

The re-crystallized montelukast cycloocyl ammonium salt (690 g) was placed in a clean, dry reactor with dichloromethane (5.8 L) and stirred for 15 minutes at room temperature. A 5M citric acid solution (3.45 L) was added and the mixture stirred for 30 minutes at room temperature to afford a two-phase system. The layers were separated and the organic layer (containing the montelukast acid) was washed three times each with 3 liters of water. Water (4.7 liters) then was added to the organic phase and a solution 1M NaOH (785 mL) was added drop-wise. The organic solvent was distilled off. The pH of the resulting aqueous layer was measured at 8.65. The aqueous layer was spray-dried to obtain montelukast sodium salt having a purity of 99.8% and containing less than 0.1% of compound (VI) (as measured by HPLC). No traces of compound (VIII) were detected in the sample.

### Example 4

### Preparation of Montelukast Cyclohexylamine Salt

A three-necked flask equipped with a thermometer, a nitrogen inlet and a magnetic stirrer was charged at room temperature with 3 g (0.006 moles) of compound (II) in 16 mL of anhydrous THF under stirring and cooled to about −20° C. N,N-diisopropylethylamine (DIEA; 2.6 mL; 0.015 moles) was added in portions followed by addition of 1 mL (0.013 moles) of methanesulfonyl chloride in portions, and stirring was maintained at about −20° C. for about 2 hours. The cold suspension containing the product compound (III), was filtered at −20° C. and the cake was washed with cold anhydrous THF.

Another three-necked flask equipped with a thermometer, a nitrogen inlet and a magnetic stirrer was charged at room temperature with 1.8 g (0.0123 moles) of compound (VII) and 16 mL of N,N-dimethylformamide (DMF) under stirring and under nitrogen atmosphere to obtain a solution. Sodium hydroxide (1.8 mL of 47% solution; 0.032 moles) was added in portions and stirring was maintained for 1 hour to afford a suspension. The solution of compound (III) in about 16 mL THF, which was kept at −20° C., was added in portions to the solution of compound (VII) at room temperature. After completing the addition, the mixture was stirred for 2 hours and reaction completion was checked by HPLC. Ethyl acetate (43 mL) and 43 mL of 5% sodium chloride solution were added to the reaction mixture, and the mixture was stirred for 20 minutes. Then, the layers were separated and the upper organic layer was washed with 43 ml of 5% sodium chloride solution, and the layers were separated. Tartaric acid (28 mL of 0.5 M solution) was added to the upper layer and the layers were separated. The upper layer was washed with 14 mL of water and again separated. The organic layer was distilled to dryness to afford an oily residue. Ethyl acetate (34 mL) was added to the residue and the mixture was distilled to dryness to afford 3.8 g of an oily residue. Ethyl acetate (34 mL) was added to the residue under stirring to obtain a solution. Cyclohexylamine (0.8 mL) was added and stirring was maintained for few minutes and the solution was seeded with crystalline montelukast acid cyclohexyl ammonium salt. Stirring was maintained at room temperature to afford a suspension, which was filtered off to obtain a cake. The cake was washed with ethyl acetate and dried at 40° C. in vacuum to afford 2.7 g of dry crude montelukast acid cyclohexyl ammonium salt in 65% yield. The HPLC purity was 95%.

### Example 5

### Preparation of Montelukast Cycloheptylamine Salt

A three-necked flask equipped with a thermometer, a nitrogen inlet and a magnetic stirrer was charged at room temperature with 3 g (0.006 moles) of compound (II) in 16 mL of anhydrous THF under stirring and cooled to about −20° C. N,N-diisopropylethylamine (2.6 mL; 0.015 moles) was added in portions followed by addition of 1 mL (0.013 moles) of methanesulfonyl chloride in portions, and stirring was maintained at about −20° C. for about 2 hours. The cold suspension containing the compound (III), was filtered at −20° C. and the cake was washed with cold anhydrous THF.

Another three-necked flask equipped with a thermometer, a nitrogen inlet and a magnetic stirrer was charged at room temperature with 1.8 g (0.0123 moles) of compound (VII) and 16 mL of N,N-dimethylformamide (DMF) under stirring and under nitrogen atmosphere to obtain a solution. Sodium hydroxide pellets (1.4 g; 0.035 moles) were added in portions and stirring was maintained for 1 hour to afford a suspension. The solution of compound (III) in about 16 mL THF, which was kept at −20° C., was added in portions to the solution of compound (VII) at room temperature. After completing the addition, the mixture was stirred for 2 hours and reaction completion was checked by HPLC. Ethyl acetate (43 mL) and 43 mL of 5% sodium chloride solution were added to the reaction mixture, and the mixture was stirred for 20 minutes. The layers were separated, and the upper organic layer was washed with 43 mL of 5% sodium chloride solution, and the layers were separated again. Tartaric acid (28 mL of 0.5 M solution) was added to the upper layer and the layers were separated. The upper layer was washed with 14 mL of water and again separated. The organic layer was distilled to dryness to afford an oily residue. Ethyl acetate (34 mL) was added to the residue and the mixture was distilled to dryness to afford 3.8 g of an oily residue. Ethyl acetate (34 mL) was added to the residue under stirring to obtain a solution. Cycloheptylamine (0.89 mL) was added and stirring was maintained for few minutes and the solution was seeded with crystalline montelukast acid cycloheptyl ammonium salt. Stirring was maintained at room temperature to afford a suspension, which was filtered off to obtain a cake. The cake was washed with ethyl acetate and dried at 40° C. in vacuum to afford 2.7 g of dry crude montelukast acid cycloheptyl ammonium salt in 65% yield. The HPLC purity was 98%.

### Example 6

### Preparation of Compound (VI)

A reaction vessel was charged with 2 g (0.0034 moles) of montelukast acid in 30 mL of toluene under stirring and the suspension was heated to reflux to afford a solution. Maleic acid (0.92 g; 0.0079 moles) was added, and reflux was maintained for about 4 hours. A sample was withdrawn and checked by HPLC to ensure that no more than 1% of the starting material was present. The reaction mixture was cooled to about 25° C. and seeded with compound (VI). Then, stirring was maintained for about 1 hour at about 25° C., and for about 1 hour at about 5° C. The mixture was filtered, washed with cold toluene and dried in vacuum to afford 1.4 g of the desired product in 70% yield, having a purity of 99.5% (by HPLC).

What is claimed:

1. A process for preparing montelukast acid or a salt thereof comprising the steps of:
(a) admixing a base and a compound (II), in a molar ratio of the base to compound (II) of about 1.1:1 to about 2:1, and cooling the resulting mixture to a temperature below 20° C.;
(b) admixing the mixture of step (a) with methanesulfonyl chloride to obtain a compound (III), wherein the molar ratio of methanesulfonyl chloride to compound (II) is about 1.05:1 to about 1.5:1; and
(c) admixing the compound (III) and a compound (VII), optionally in the presence of a base, to obtain montelukast or a salt thereof;
wherein the amount of a compound (VI) formed in the process is less than 0.1% by weight.

2. The process of claim 1 wherein the molar ratio of methanesulfonyl chloride to compound (II) is about 1.1:1 to about 1.45:1.

3. The process of claim 1 wherein the molar ratio of methanesulfonyl chloride to compound (II) is about 1.35:1 to about 1.45:1.

4. The process of claim 1 wherein the methanesulfonyl chloride is added to the cooled mixture of step (a).

5. The process of claim 4 wherein the methansulfonyl chloride is added in at least two portions.

6. The process of claim 1 wherein the mixture of step (a) is cooled to a temperature below about 20° C.

7. The process of claim 1 wherein the mixture of step (a) is cooled to a temperature at or below about 0° C.

8. The process of claim 1 wherein the base of step (a) is selected from the group consisting of triethylamine, tripropylamine, triisopropylamine, tributylamine, triisobutylamine, N,N-diisopropylethylamine, N,N-dimethylaniline, and mixtures thereof.

9. The process of claim 1 wherein the molar ratio of the base to compound (II) in step (a) is about 1.2:1 to about 1.8:1.

10. The process of claim 1 wherein the molar ratio of the base to compound (II) in step (a) is about 1.3:1 to about 1.5:1.

11. The process of claim 1 wherein the base in step (a) comprises N,N-diisopropylethylamine.

12. The process of claim 1 wherein the admixing of compound (III) and compound (VII) of step (c) comprises the steps of:
(1) mixing the compound (VII) and an organic solvent;
(2) adding a base to the mixture of step (1); and
(3) adding the compound (III) to the mixture of step (2), wherein the adding is drop-wise or in at least two portions.

13. The process of claim 12 wherein the organic solvent is selected from the group consisting of N,N-dimethylformamide, N,N-dimethylacetamide, dimethyl sulfoxide, N-methyl-2-pyrrolidone, tetrahydrofuran, 2-methyltetrahydrofuran, acetonitrile, acetone, and mixtures thereof.

14. The process of claim 12 wherein the organic solvent comprises N-methyl-2-pyrrolidone.

15. The process of claim 12 wherein step (1) further comprises adding a cosolvent to form a suspension.

16. The process of claim 15 wherein the co-solvent comprises water.

17. The process of claim 15 wherein the co-solvent is about 5% by volume of the organic solvent.

18. The process of claim 12 wherein the base is selected from the group consisting of lithium hydroxide, potassium hydroxide, sodium hydroxide, sodium carbonate, potassium carbonate, sodium bicarbonate, potassium bicarbonate, and mixtures thereof.

19. The process of claim 12 wherein the base comprises sodium hydroxide.

20. The process of claim 19 wherein the molar ratio of sodium hydroxide to compound (VII) is about 1.1:1 to about 2.5:1.

21. The process of claim 1 further comprising admixing an organic acid and the reaction product of step (c) to obtain montelukast acid.

22. The process of claim 21 wherein the organic acid is selected from the group consisting of acetic acid, propionic acid, oxalic acid, benzoic acid, maleic acid, malonic acid, fumaric acid, tartaric acid, malic acid, citric acid, and mixtures thereof.

23. The process of claim 21 wherein the organic acid comprises tartaric acid or citric acid.

24. The process of claim 1 further comprising admixing an amine and the reaction product of step (c) to obtain to an ammonium salt of montelukast.

25. The process of claim 24 further comprising seeding the admixture of montelukast acid and the amine with a crystal of the ammonium salt of montelukast.

26. The process of claim 24 further comprising purifying the ammonium salt of montelukast.

27. The process of claim 24 wherein the amine is selected from the group consisting of cyclohexylamine, cyclopentylamine, cycloheptylamine, cyclooctylamine, cyclododecylamine, phenethylamine, and mixtures thereof.

28. The process of claim 24 wherein the amine comprises cyclooctylamine.

29. The process of claim 24 further comprising crystallizing the ammonium salt of montelukast.

30. The process of claim 29 wherein the crystallizing comprises adding an organic solvent to the ammonium salt of montelukast, and the organic solvent is selected from the group consisting of methanol, methyl acetate, ethyl acetate, n-propyl acetate, isopropyl acetate, n-butyl acetate, isobutyl acetate, toluene, and mixtures thereof.

31. The process of claim 30 wherein the organic solvent comprises toluene optionally containing and up to 5% by volume methanol.

32. The process of claim 24 further comprising the steps of:
(1) admixing the ammonium salt of montelukast, an acid, an organic solvent, and water;
(2) separating the water from the admixture of step (1);
(3) optionally evaporating at least a portion of the organic solvent;
(4) adding a sodium base and water to the admixture of step (2) or step (3);
(5) separating the organic solvent from the admixture of step (4); and
(6) drying the admixture of step (5) to obtain the montelukast sodium.

33. The process of claim 32, wherein the drying comprises spray drying.

34. The process of claim 32, wherein the acid of step (1) is an organic acid selected from the group consisting of acetic acid, propionic acid, oxalic acid, benzoic acid, maleic acid, malonic acid, fumaric acid, tartaric acid, malic acid, citric acid, and mixtures thereof.

35. The process of claim 32, wherein the acid of step (1) comprises tartaric acid or citric acid.
